(19) United States
(12) Patent Application Publication
(10) Pub. No.: US 2025/0122569 A1
(43) Pub. Date: Apr. 17, 2025

(54) DIAGNOSIS AND TREATMENT OF DISEASES AND CONDITIONS OF THE INTESTINAL TRACT

(71) Applicant: Flagship Pioneering Innovations VI, LLC, Cambridge, MA (US)

(72) Inventors: Douglas Gowers COLE, Wayland, MA (US); Jason PARK, West Newton, MA (US); Sabrina Yusang YANG, Cambridge, MA (US); Boris HAYETE, Needham, MA (US); Andrew John GASPARRINI, Boston, MA (US)

(21) Appl. No.: 18/694,601

(22) PCT Filed: Sep. 23, 2022
(86) PCT No.: PCT/US2022/076931

§ 371 Date: Mar. 22, 2024

Related U.S. Application Data
(60) Provisional application No. 63/247,448, filed on Sep. 23, 2021.

(51) Int. Cl. C12Q 1/6883

(52) U.S. Cl. CPC C12Q 1/6883; C12Q 2600/106; C12Q 2600/158

(57) ABSTRACT

The invention relates to methods of predicting a patient's responsiveness to treatment (e.g., vedolizumab treatment) for inflammatory bowel disease (IBD).

### SEQUENCE LISTING

The instant application contains a Sequence Listing which has been submitted electronically in XML format and is hereby incorporated by reference in its entirety. Said XML copy, created on Sep. 15, 2022, is named 51373-018WO1_Sequence_Listing_9_15_22.xml and is 26,504,024 bytes in size.

### FIELD OF THE INVENTION

The invention relates to methods of predicting a patient's responsiveness to treatment (e.g., vedolizumab treatment) for inflammatory bowel disease (IBD).

### BACKGROUND

Mammals are colonized by microorganisms in the gastrointestinal (GI) tract, on the skin, and in other epithelial and tissue niches. The gastrointestinal tract of a healthy individual harbors an abundant and diverse microbial community. It is a complex system, providing an environment or niche for a community of many different species or organisms, including diverse strains of bacteria. Hundreds of different species may form a commensal community in the GI tract in a healthy person, and this complement of organisms evolves from the time of birth and is believed to form a functionally mature microbial population by about 3 years of age. Interactions between microbial strains in these populations and between microorganisms and the host, e.g., interactions with the host's immune system, shape the community structure, with availability of and competition for resources affecting the distribution of microorganisms.

A healthy microbiome may provide a subject with multiple benefits, including colonization resistance to a broad spectrum of pathogens, essential nutrient biosynthesis and absorption, and immune stimulation that plays a role in maintaining a healthy gut epithelium and appropriately controlled systemic immunity. Conversely, an unhealthy (e.g., dysregulated) microbiome may be associated with a disease state.

There is a need for methods for addressing problems in healthcare through assessment of the microbiome.

### SUMMARY OF THE INVENTION

In one aspect, the disclosure features a method of identifying a subject having an inflammatory bowel disease (IBD) who is likely to respond to treatment with an anti-integrin or other IBD therapy, the method comprising determining a level of one or more of SEQ ID NOs: 1-1171 in a sample from the subject, wherein a level of one or more of SEQ ID NOs: 1-1171 that is changed relative to a respective reference level for SEQ ID NOs: 1-1171 indicates that the subject is likely to respond to treatment with the anti-integrin or other IBD therapy.

In another aspect, the disclosure features a method for determining that a subject having an IBD is likely to respond to treatment with an anti-integrin therapy or other IBD therapy, comprising the steps of (a) measuring the nucleic acid level of one or more of SEQ ID NOs: 1-1171 in a sample collected from the subject using amplification and one or more pairs of primers specific for each of the one or more of SEQ ID NOs: 1-1171; and (b) using the amplification results to determine whether the level of one or more of SEQ ID NOs: 1-1171 is changed relative to a respective reference level for SEQ ID NOs: 1-1171, thereby determining that the subject is likely to respond to treatment with an anti-integrin therapy or other IBD therapy.

In some embodiments, a level of one or more of SEQ ID NOs: 1-1171 is changed relative to the respective reference level for SEQ ID NO: 1-1171 in the sample from the subject and the method further comprises administering an anti-integrin or other IBD therapy to a subject.

In some embodiments, the method comprises determining or measuring a level of at least two, at least three, at least four, at least five, at least six, at least seven, at least eight, at least nine, at least ten, at least fifteen, at least 20, at least 30, at least 40, at least 50, at least 60, at least 70, at least 80, at least 90, at least 100, at least 110, at least 120, at least 130, at least 140, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, or at least 1150 of SEQ ID NOs: 1-1171 in the sample from the subject.

In some embodiments, the method comprises determining or measuring a level of all 1171 of SEQ ID NOs: 1-1171 in the sample from the subject.

In some embodiments, the anti-integrin therapy targets integrin α₄β₇. In some embodiments, the anti-integrin therapy is vedolizumab.

In some embodiments, the method further comprises treatment comprising fecal microbiota transplantation (FMT).

In some embodiments, the reference level is a pre-assigned level.

In some embodiments, the reference level is a level in a set of samples from a reference population. In some embodiments, the reference population is a population of healthy subjects.

In some embodiments, the level of each of SEQ ID NOs: 1-1171 that is determined or measured in the sample from the subject is a nucleic acid level. In some embodiments, the nucleic acid level is a DNA level.

In some embodiments, the change is a decrease relative to the reference level. In some embodiments, the levels of at least 10%, at least 20%, at least 30%, at least 40%, at least 50%, at least 60%, at least 70%, at least 80%, at least 90%, or more than 90% of the sequences for which a level is determined or measured are decreased relative to a respective reference level for the sequence.

In some embodiments, the change is an increase relative to the reference level. In some embodiments, the levels of at least 10%, at least 20%, at least 30%, at least 40%, at least 50%, at least 60%, at least 70%, at least 80%, at least 90%, or more than 90% of the sequences for which a level is determined or measured are increased relative to a respective reference level for the sequence.

In some embodiments, the sample comprises a sample of the microbiota of the subject.

In some embodiments, the sample is a fecal sample.

In another aspect, the disclosure features a kit for identifying a subject having an inflammatory bowel disease (IBD) who is likely to respond to treatment with an anti-integrin or other IBD therapy, the kit comprising: (a) polypeptides or polynucleotides capable of determining the level of one or more of SEQ ID NOs: 1-1171 in a sample from the subject; and (b) instructions for use of the polypeptides or polynucleotides to determine the level of one or more of SEQ ID NOs: 1-1171 in a sample from the subject, wherein a level of one or more of SEQ ID NOs: 1-1171 that is changed relative to a respective reference level for SEQ ID NOs: 1-1171 indicates that the subject is likely to respond to treatment with the anti-integrin or other IBD therapy.

In some embodiments, the kit comprises polypeptides or polynucleotides capable of determining the level of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or more than 99% of SEQ ID NOs: 1-1171 in a sample from the subject.

In another aspect, the disclosure features a method of identifying a subject having an inflammatory bowel disease (IBD) who is likely to respond to treatment with an anti-integrin or other IBD therapy, the method comprising determining a level of each of SEQ ID NOs: 1-1171 in a sample from the subject, wherein a level of at least 50% of SEQ ID NOs: 1-1171 that is changed relative to a respective reference level for SEQ ID NOs: 1-1171 indicates that the subject is likely to respond to treatment with an anti-integrin or other IBD therapy.

In some embodiments, a level of at least 50% of SEQ ID NOs: 1-1171 is changed relative to a respective reference level for SEQ ID NOs: 1-1171 in the sample from the subject and the method further comprises administering an anti-integrin or other IBD therapy to a subject.

In some embodiments, a level of at least 60%, 70%, 80%, 90%, or 95% of SEQ ID NOs: 1-1171 that is changed in a sample from the subject relative to a respective reference level for SEQ ID NOs: 1-1171 indicates that the subject is likely to respond to treatment with an anti-integrin or other IBD therapy.

In some embodiments, the anti-integrin therapy targets integrin α₄β₇. In some embodiments, the anti-integrin therapy is vedolizumab.

In another aspect, the disclosure features a method of treating a subject having an IBD using an anti-integrin therapy or other IBD therapy, wherein the subject was determined to be likely to respond to treatment with an anti-integrin therapy or other IBD therapy by any of the methods provided herein.

In another aspect, the disclosure features a method of treating a subject having an IBD using an anti-integrin therapy or other IBD therapy, the method comprising the steps of (a) measuring the nucleic acid level of one or more of SEQ ID NOs: 1-1171 in a sample collected from the subject using amplification and one or more pairs of primers specific for each of the one or more of SEQ ID NOs: 1-1171; (b) using the amplification results to determine whether the level of one or more of SEQ ID NOs: 1-1171 is changed relative to a respective reference level for SEQ ID NOs: 1-1171, thereby determining an increased likelihood of response to treatment with an anti-integrin therapy or other IBD therapy for the subject; and (c) treating a subject who has been determined to have an increased likelihood of response to treatment with an anti-integrin therapy or other IBD therapy with the anti-integrin therapy or other IBD therapy.

In some embodiments, the nucleic acid level of the one or more of SEQ ID NOs: 1-1171 in the sample collected from the subject are measured using amplification and one or more pairs of primers specific for each of the one or more of SEQ ID NOs: 1-1171, and the amplification results are used to determine whether the level of one or more of SEQ ID NOs: 1-1171 is changed relative to a respective reference level for SEQ ID NOs: 1-1171.

In some embodiments, the method comprises measuring the nucleic acid level of at least two, at least three, at least four, at least five, at least six, at least seven, at least eight, at least nine, at least ten, at least fifteen, at least 20, at least 30, at least 40, at least 50, at least 60, at least 70, at least 80, at least 90, at least 100, at least 110, at least 120, at least 130, at least 140, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, or at least 1150 of SEQ ID NOs: 1-1171 in the sample from the subject. In some embodiments, the method comprises measuring the nucleic acid level of all 1171 of SEQ ID NOs: 1-1171 in the sample from the subject.

In some embodiments, the reference level is a pre-assigned level. In some embodiments, the reference level is a level in a set of samples from a reference population. In some embodiments, the reference population is a population of healthy subjects.

In some embodiments, the nucleic acid level is a DNA level.

In some embodiments, the change is a decrease relative to the reference level. In some embodiments, the levels of at least 10%, at least 20%, at least 30%, at least 40%, at least 50%, at least 60%, at least 70%, at least 80%, at least 90%, or more than 90% of the sequences for which a level is measured are decreased relative to a respective reference level for the sequence.

In some embodiments, the change is an increase relative to the reference level. In some embodiments, the levels of at least 10%, at least 20%, at least 30%, at least 40%, at least 50%, at least 60%, at least 70%, at least 80%, at least 90%, or more than 90% of the sequences for which a level is measured are increased relative to a respective reference level for the sequence.

In some embodiments, the sample comprises a sample of the microbiota of the subject. In some embodiments, the sample is a fecal sample.

In another aspect, the disclosure features a method of treating a subject having an IBD, the method comprising the steps of (a) measuring the nucleic acid level of each of SEQ ID NOs: 1-1171 in a sample collected from the subject, wherein a level of at least 50% of SEQ ID NOs: 1-1171 that is changed relative to a respective reference level for SEQ ID NOs: 1-1171 indicates that the subject is likely to respond to treatment with an anti-integrin therapy or other IBD therapy; and (b) treating a subject who has been determined to be likely to respond to treatment with an anti-integrin therapy or other IBD therapy with the anti-integrin therapy or other IBD therapy.

In some embodiments, the nucleic acid levels of each of SEQ ID NOs: 1-1171 in the sample collected from the subject are measured using amplification and one or more pairs of primers specific for each of SEQ ID NOs: 1-1171, and the amplification results are used to determine whether the level of each of SEQ ID NOs: 1-1171 is changed relative to a respective reference level for SEQ ID NOs: 1-1171.

### BRIEF DESCRIPTION OF THE DRAWING

FIG. 1 is a graph showing the out-of-cohort performance (as percent specificity and sensitivity) of metagenomic markers for distinguishing patients who were to be responders vs. non-responders to treatment with the anti-integrin therapy vedolizumab.

### DETAILED DESCRIPTION OF THE INVENTION

### Definitions

The term “changed,” as used herein, refers to an observable difference in the level of a marker in a subject (e.g., in a sample from the subject), as determined using techniques and methods known in the art for the measurement of the marker. A marker level that is changed in a subject may result in a difference of at least 1% (e.g., at least 5%, 10%, 15%, 20%, 25%, 30%, 35%, 40%, 45%, 50%, 55%, 60%, 65%, 70%, 75%, 80%, 85%, 90%, 95%, or 100%, or at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) more or less than a reference level (e.g., a level from a healthy subject or a level prior to treatment) (e.g., up to 100% or up to 100-fold relative to the reference level). In some embodiments, the change is an increase in the level of a marker in a subject. Increasing the marker level in a subject may result in an increase of at least 1% (e.g., at least 5%, 10%, 15%, 20%, 25%, 30%, 35%, 40%, 45%, 50%, 55%, 60%, 65%, 70%, 75%, 80%, 85%, 90%, 95%, 98%, 99%, or 100%, or at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) relative to the reference level (e.g., up to 100% or up to 100-fold relative to the reference level). In other embodiments, the change is a decrease the level of a marker in a subject. Decreasing the marker level in a subject may result in a decrease of at least 1% (e.g., at least 5%, 10%, 15%, 20%, 25%, 30%, 35%, 40%, 45%, 50%, 55%, 60%, 65%, 70%, 75%, 80%, 85%, 90%, 95%, or 100%, or at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) relative to the reference level (e.g., up to 100% or up to 100-fold relative to the reference level).

In embodiments in which a level is increased or decreased (or reduced) in a subject following a step of administering a therapy described herein, the increase or decrease may take place and/or be detectable within a range of time following the administration (e.g., within six hours, 24 hours, 3 days, a week or longer), and may take place and/or be detectable after one or more administrations (e.g., after 2, 3, 4, 5, 6, 7, 8, 9, 10, or more administrations, e.g., as part of a dosing regimen for the subject).

In some embodiments, the change in the level of a portion of the markers analyzed is an increase, while the change in the level of another portion of the markers analyzed is a decrease. In some embodiments, the change in at least 10%, 20%, 30%, 40%, 50%, 60%, 70%, 80%, 90%, 95%, or more (e.g., 100%) of the markers analyzed is an increase relative to a reference level. In some embodiments, the change in at least 10%, 20%, 30%, 40%, 50%, 60%, 70%, 80%, 90%, 95%, or more (e.g., 100%) of the markers analyzed is a decrease relative to a reference level.

The term “pharmaceutical composition,” as used herein, represents a composition formulated with a pharmaceutically acceptable excipient. For example, a “pharmaceutical composition” can be a composition that is manufactured or sold with the approval of a governmental regulatory agency as part of a therapeutic regimen for the treatment of a disease, disorder, or condition in a mammal, intended for such use, or in development for such use. In some examples, the pharmaceutical composition is a pre-approved composition.

The term “subject,” as used herein, represents a human or non-human animal (e.g., a mammal).

“Treatment” and “treating,” as used herein, refer to the medical management of a subject with the intent to improve, ameliorate, stabilize, prevent, or cure a disease, disorder, or condition. This term includes active treatment (treatment directed to improve the disease, disorder, or condition); causal treatment (treatment directed to the cause of the associated disease, disorder, or condition); palliative treatment (treatment designed for the relief of symptoms of the disease, disorder, or condition); preventative treatment (treatment directed to minimizing or partially or completely inhibiting the development of the associated disease, disorder, or condition); and supportive treatment (treatment employed to supplement another therapy).

The term “inflammatory bowel disease” or “IBD,” as used herein, refers to a condition of the bowel, e.g., the small intestine, large intestine, mouth, esophagus, stomach, rectum, and/or anus, that is characterized by inflammation. Examples of IBD include ulcerative colitis (UC) and Crohn's disease. Other examples include microscopic colitis (e.g., collagenous colitis or lyphocytic colitis), diversion colitis, Behçet's disease, or indeterminate colitis.

### I. Methods for Predicting Responsiveness to Therapy in Inflammatory Bowel Disease

The invention is based, in part, on the discovery that levels of gut microbiome biomarkers (i.e., markers of bacterial origin), which may be referred to as co-evolved molecules, can be used to identify patients having an inflammatory bowel disease (IBD) who are likely to respond to treatment with an anti-integrin or other IBD therapy. Accordingly, the disclosure provides methods of diagnosing, treating, and monitoring subjects (e.g., human patients) based on this discovery.

### Diagnostic Methods and Methods of Treatment

In some aspects, the disclosure features a method of identifying a subject having an inflammatory bowel disease (IBD) who is likely to respond to treatment with an anti-integrin or other IBD therapy, the method comprising determining a level of one or more of SEQ ID NOs: 1-1171 in a sample from the subject, wherein a level of one or more of SEQ ID NOs: 1-1171 that is changed relative to a respective reference level for SEQ ID NOs: 1-1171 indicates that the subject is likely to respond to treatment with the anti-integrin or other IBD therapy. SEQ ID NOs: 1-1171 are bacterial sequences.

In some aspects, the disclosure features a method for determining that a subject having an IBD is likely to respond to treatment with an anti-integrin therapy or other IBD therapy, comprising the steps of (a) measuring the nucleic acid level of one or more of SEQ ID NOs: 1-1171 in a sample collected from the subject using amplification and one or more pairs of primers specific for each of the one or more of SEQ ID NOs: 1-1171; and (b) using the amplification results to determine whether the level of one or more of SEQ ID NOs: 1-1171 is changed relative to a respective reference level for SEQ ID NOs: 1-1171, thereby determining that the subject is likely to respond to treatment with an anti-integrin therapy or other IBD therapy.

In some aspects, the disclosure features a method of treating a subject having an IBD using an anti-integrin therapy or other IBD therapy, wherein the subject was determined to be likely to respond to treatment with an anti-integrin therapy or other IBD therapy by any of the methods provided herein.

In some aspects, the disclosure features a method of treating a subject having an IBD using an anti-integrin therapy or other IBD therapy, the method comprising the steps of (a) measuring the nucleic acid level of one or more of SEQ ID NOs: 1-1171 in a sample collected from the subject using amplification and one or more pairs of primers specific for each of the one or more of SEQ ID NOs: 1-1171; (b) using the amplification results to determine whether the level of one or more of SEQ ID NOs: 1-1171 is changed relative to a respective reference level for SEQ ID NOs: 1-1171, thereby determining an increased likelihood of response to treatment with an anti-integrin therapy or other IBD therapy for the subject; and (c) treating a subject who has been determined to have an increased likelihood of response to treatment with an anti-integrin therapy or other IBD therapy with the anti-integrin therapy or other IBD therapy.

In some aspects, the disclosure features a method of treating a subject having an IBD, the method comprising the steps of (a) measuring the nucleic acid level of each of SEQ ID NOs: 1-1171 in a sample collected from the subject, wherein a level of at least 50% of SEQ ID NOs: 1-1171 that is changed relative to a respective reference level for SEQ ID NOs: 1-1171 indicates that the subject is likely to respond to treatment with an anti-integrin therapy or other IBD therapy; and (b) treating a subject who has been determined to be likely to respond to treatment with an anti-integrin therapy or other IBD therapy with the anti-integrin therapy or other IBD therapy.

In some embodiments, the sample comprises a sample of the microbiota (e.g., the gut microbiota) of the subject. In some embodiments, the sample is a fecal sample. In some embodiments, the sample is a colon or rectal biopsy.

In some embodiments, the method comprises determining or measuring a level of at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 325, at least 350, at least 375, at least 400, at least 425, at least 450, at least 475, at least 500, at least 525, at least 550, at least 575, at least 600, at least 625, at least 650, at least 675, at least 700, least 725,at least 750, at least 775, at least 800, at least 825, at least 850, at least 875, at least 900, at least 925, at least 950, at least 975, at least 1000, at least 1025, at least 1050, at least 1075, at least 1100, or at least 1150 of SEQ ID NOs: 1-1171 in a sample from the subject, e.g., comprises determining a level of 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-200, 200-205, 205-210, 210-215, 215-220, 220-225, 225-230, 230-235, 235-240, 240-245, 245-250, 250-255, 255-260, 260-265, 265-270, 270-275, 275-280, 280-285, 285-290, 290-295, 295-300, 300-305, 305-310, 310-315, 315-320, 325-350, 350-375, 375-400, 400-425, 425-450, 450-475, 475-500, 500-525, 525-550, 550-575, 575-600, 600-625, 625-650, 650-675, 675-700, 700-725, 725-750, 750-775, 775-800, 800-825, 825-850, 850-875, 875-900, 900-925, 925-950, 950-975, 975-1000, 1000-1025, 1025-1050, 1050-1075, 1075-1100, 1100-1125, 1125-1150, or 1150-1170 of SEQ ID NOs: 1-1171 in the sample from the subject. In some embodiments, the method comprises determining a level of all 1171of SEQ ID NOs: 1-1171 in a sample from the subject.

In some embodiments, a change in the a level of at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 325, at least 350, at least 375, at least 400, at least 425, at least 450, at least 475, at least 500, at least 525, at least 550, at least 575, at least 600, at least 625, at least 650, at least 675, at least 700, least 725, at least 750, at least 775, at least 800, at least 825, at least 850, at least 875, at least 900, at least 925, at least 950, at least 975, at least 1000, at least 1025, at least 1050, at least 1075, at least 1100, or at least 1150 of SEQ ID NOs: 1-1171 in a sample from the subject, e.g., a change in the level of 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-200, 200-205, 205-210, 210-215, 215-220, 220-225, 225-230, 230-235, 235-240, 240-245, 245-250, 250-255, 255-260, 260-265, 265-270, 270-275, 275-280, 280-285, 285-290, 290-295, 295-300, 300-305, 305-310, 310-315, 315-320, 325-350, 350-375, 375-400, 400-425, 425-450, 450-475, 475-500, 500-525, 525-550, 550-575, 575-600, 600-625, 625-650, 650-675, 675-700, 700-725, 725-750, 750-775, 775-800, 800-825, 825-850, 850-875, 875-900, 900-925, 925-950, 950-975, 975-1000, 1000-1025, 1025-1050, 1050-1075, 1075-1100, 1100-1125, 1125-1150, or 1150-1170 of SEQ ID NOs: 1-1171 in the sample from the subject relative to a respective reference level for SEQ ID NOs: 1-1171 indicates that the subject is likely to respond to treatment with an anti-integrin or other IBD therapy. In some embodiments, a change in the level of all 1171 of SEQ ID NOs: 1-1171 relative to a respective reference level for SEQ ID NOs: 1-1171 indicates that the subject is likely to respond to treatment with an anti-integrin or other IBD therapy.

In some embodiments, a change in the level of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 95%, at least 98%, at least 99%, or 100% of the total number of SEQ ID NOs: 1-1171 measured in a sample from the subject relative to a respective reference level for SEQ ID NOs: 1-1171 indicates that the subject is likely to respond to treatment with the anti-integrin or other IBD therapy.

In some embodiments, a threshold number of one or more of SEQ ID NOs: 1-1171 is changed relative to the respective reference level for SEQ ID NO: 1-1171 in the sample from the subject (e.g., a number of SEQ ID NOs: 1-1171 that has been determined to indicate that the patient is likely to respond to treatment with an anti-integrin or other IBD therapy, e.g., a level of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 325, at least 350, at least 375, at least 400, at least 425, at least 450, at least 475, at least 500, at least 525, at least 550, at least 575, at least 600, at least 625, at least 650, at least 675, at least 700, least 725, at least 750, at least 775, at least 800, at least 825, at least 850, at least 875, at least 900, at least 925, at least 950, at least 975, at least 1000, at least 1025, at least 1050, at least 1075, at least 1100, or at least 1150 of SEQ ID NOs: 1-1171 in a sample from the subject (e.g., a level of 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-200, 200-205, 205-210, 210-215, 215-220, 220-225, 225-230, 230-235, 235-240, 240-245, 245-250, 250-255, 255-260, 260-265, 265-270, 270-275, 275-280, 280-285, 285-290, 290-295, 295-300, 300-305, 305-310, 310-315, 315-320, 325-350, 350-375, 375-400, 400-425, 425-450, 450-475, 475-500, 500-525, 525-550, 550-575, 575-600, 600-625, 625-650, 650-675, 675-700, 700-725, 725-750, 750-775, 775-800, 800-825, 825-850, 850-875, 875-900, 900-925, 925-950, 950-975, 975-1000, 1000-1025, 1025-1050, 1050-1075, 1075-1100, 1100-1125, 1125-1150, or 1150-1170) of SEQ ID NOs: 1-1171 is changed relative to the respective reference level for SEQ ID NO: 1-1171 in the sample from the subject and the method further comprises administering an anti-integrin or other IBD therapy to a subject.

In some aspects, the anti-integrin therapy targets integrin daß7. In some aspects, the anti-integrin therapy is vedolizumab. In some aspects, the method further comprises treatment by fecal microbiota transplant (FMT).

IBD therapies that may be used in the invention further include a biologic therapy (e.g., vedolizumab (ENTYVIO®), infliximab (REMICADE®), adalimumab (HUMIRA®), golimumab (SIMPONI®), certolizumab (CIMZIA®), risankizumab (SKYRIZI®), or ustekinumab (STELARA®)); an anti-inflammatory agent (e.g., a corticosteroid or an aminosalicylate, such as mesalamine (e.g., DELZICOL®, ROWASA®, others), balsalazide (COLAZAL®), or olsalazine (DIPENTUM®)); an antibiotic, e.g., ciprofloxacin (cipro) or metronidazole (Flagyl); or an immune system suppressor (e.g., azathioprine (AZASAN®, IMURAN®), mercaptopurine (PURINETHOL®, PURIXAN®), methotrexate (TREXALL®), tofacitinib (XELJANZ®), upadacitinib (RINVOQ®), or ozanimod (ZEPOSIA®)). Any one or more of these therapies may optionally be used in any of the methods described herein as employing an IBD therapy.

In some embodiments, the respective reference level for SEQ ID NOs: 1-1171 is a pre-assigned level of one of SEQ ID NOs: 1-1171.

In some embodiments, the respective reference level for SEQ ID NOs: 1-1171 is a level in a set of samples from a reference population, e.g., a population of healthy subjects (e.g., a population of subjects not having an inflammatory bowel disease (IBD) (e.g., not having ulcerative colitis and/or not having Crohn's disease) and/or a population of subjects having a healthy gut microbiome). In some embodiments, the reference subjects are human subjects.

In some embodiments, the level of each of SEQ ID NOs: 1-1171 that is determined or measured in a sample from the subject is a nucleic acid level, e.g., a DNA level or an RNA level. In some embodiments, the nucleic acid level is a DNA level, which may be detected, e.g., using a PCR-based method. In other embodiments, detection of a level of one or more of SEQ ID NOs: 1-1171 can optionally comprise, for example, detection of RNA levels, which can be achieved by, e.g., RT-PCR, RNA-Seq, and/or methods including the use of microarrays, as are known in the art. In other embodiments, the methods can focus on the detection of protein levels, which can be carried out using standard approaches (e.g., immunoassay-based approaches).

In some embodiments, the change in a level of one or more of SEQ ID NOs: 1-1171 in the sample from the subject is a decrease relative to the respective reference level for SEQ ID NOs: 1-1171 (e.g., a decrease of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, e.g., a decrease of, e.g., at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) relative to the respective reference level for SEQ ID NOs: 1-1171; or a decrease of 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 0-85%, 85-90%, 90-95%, or 95-100% relative to the respective reference level for SEQ ID NOs: 1-1171).

In some aspects, the levels of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or more than 99% of the sequences for which a level is determined or measured in the sample from the subject (i.e., one or more of SEQ ID NOs: 1-1171) are decreased relative to a respective reference level for the sequence, e.g., 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 0-85%, 85-90%, 90-95%, or 95-100% of the sequences for which a level is determined or measured are decreased relative to a respective reference level for the sequence.

In some embodiments, the change in a level of one or more of SEQ ID NOs: 1-1171in the sample from the subject is an increase relative to the respective reference level for SEQ ID NOs: 1-1171 (e.g., an increase of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more than 100%, e.g., an increase of, e.g., 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold, relative to the respective level for SEQ ID NOs: 1-1171; or an increase of 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 0-85%, 85-90%, 90-95%, 95-100%, or more than 100%, relative to the respective reference level for SEQ ID NOs: 1-1171).

In some aspects, the levels of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or more than 99% of the sequences for which a level is determined or measured in the sample from the subject (i.e., one or more of SEQ ID NOs: 1-1171) are increased relative to a respective reference level for the sequence, e.g., 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 0-85%, 85-90%, 90-95%, or 95-100% of the sequences for which a level is determined or measured are increased relative to a respective reference level for the sequence.

In some aspects, a level of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of SEQ ID NOs: 1-1171 that is increased in a sample from the subject relative to a respective reference level for SEQ ID NOs: 1-1171 indicates that the subject is likely to respond to treatment with the anti-integrin or other IBD therapy.

In another aspect, the disclosure features a method of identifying a subject having an inflammatory bowel disease (IBD) who is likely to respond to treatment with an anti-integrin or other IBD therapy, the method comprising determining a level of each of SEQ ID NOs: 1-1171 in a sample from the subject, wherein a level of at least 50% of SEQ ID NOs: 1-1171 that is changed (e.g., increased or decreased) relative to a respective reference level for SEQ ID NOs: 1-1171 indicates that the subject is likely to respond to treatment with an anti-integrin or other IBD therapy.

In some aspects, a level of at least 50% of SEQ ID NOs: 1-1171 is increased relative to a respective reference level for SEQ ID NOs: 1-1171 in the sample from the subject and the method further comprises administering an anti-integrin or other IBD therapy to a subject.

In some aspects, a level of at least 60%, 70%, 80%, 90%, or 95% of SEQ ID NOS: 1-1171 that is increased in a sample from the subject relative to a respective reference level for SEQ ID NOs: 1-1171 indicates that the subject is likely to respond to treatment with an anti-integrin or other IBD therapy.

Determination of whether a difference detected is significant can be carried out using standard methods, as well as statistical analysis. In some embodiments, a difference detected is a change of at least 5%, 10%, 20%, 30%, 40%, 50%, 75%, 100%, or more, e.g., at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold, relative to a reference level.

### II. Articles of Manufacture and Kits

In another aspect of the invention, an article of manufacture or kit containing materials useful for the diagnosis, prognostic assessment, and/or treatment of individuals is provided.

In one aspect, the disclosure features a kit or article of manufacture for identifying a subject having an inflammatory bowel disease (IBD) who is likely to respond to treatment with an anti-integrin or other IBD therapy, the kit comprising (a) reagents for determining a level of one or more of SEQ ID NOs: 1-1171 in a sample from the subject (e.g., polynucleotides or polypeptides capable of use in determining the level of one or more of SEQ ID NOs: 1-1171 in a sample from the subject); and optionally (b) instructions for use of the polynucleotides or polypeptides to determine the level of one or more of SEQ ID NOs: 1-1171 in the sample from the subject, wherein a change in the level of one or more of SEQ ID NOs: 1-1171 relative to a respective reference level for SEQ ID NOs: 1-1171, as described herein, indicates that the subject is likely to respond to treatment with the anti-integrin or other IBD therapy.

In some aspects, the reagents for determining a level of one or more of SEQ ID NOs: 1-1171 in a sample from the subject comprise one or more polynucleotides (e.g., PCR primers) that hybridize to a complement of a locus of one or more of SEQ ID NOs: 1-1171 under stringent conditions and may be used to amplify all or a portion of any one or more of SEQ ID NOs: 1-1171, as described herein. In some aspects, the instructions indicate that the one or more oligonucleotides (e.g., PCR primers) may be used to evaluate the presence and/or level of one or more of SEQ ID NOs: 1-1171 in a sample from the subject and provide instructions for using the polynucleotide(s) for evaluating the presence and/or level of one or more of SEQ ID NOs: 1-1171 in the sample. In some embodiments, reagents are included for determining a level of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or more than 99% of the sequences of SEQ ID NOs: 1-1171.

For polynucleotide-based articles of manufacture or kits, the article of manufacture or kit may include, for example: (1) an oligonucleotide, e.g., a detectably labeled oligonucleotide, which hybridizes to a nucleic acid sequence encoding a protein, (2) a pair of primers useful for amplifying a nucleic acid molecule, or (3) a microarray comprising multiple oligonucleotide probes. For protein-based articles of manufacture or kits, the article of manufacture or kit may include, for example, one or more antibody-based reagents. The article of manufacture or kit can also include, e.g., a buffering agent, a preservative, or a protein-stabilizing agent. The article of manufacture or kit can further include components necessary for detecting the detectable label (e.g., an enzyme or a substrate). The article of manufacture or kit can further include components necessary for analyzing the sequence of a sample (e.g., a restriction enzyme or a buffer). The article of manufacture or kit can also contain a control sample or a series of control samples that can be assayed and compared to the test sample (e.g., a reference sample, as described herein). Each component of the article of manufacture or kit can be enclosed within an individual container and all of the various containers can be within a single package, along with instructions for interpreting the results of the assays performed using the kit.

The following examples are meant to illustrate the invention. They are not meant to limit the invention in any way.

### III. Examples

### Example 1. Identification of Markers

### A. Assembly and Annotation of Metagenomic Markers

Paired end reads from healthy Human Microbiome Project 1 (HMP1; Human Microbiome Project Consortium, Nature, 486(7402): 207-214, 2012) individuals were downloaded from the National Center for Bioinformatics (NCBI) Short Read Archive (SRA) and assembled using metaSPAdes (cab.spbu.ru/software/spades/). Metagenomic markers were annotated using antiSMASH4.0 (docs.antismash.secondarymetabolites.org/) with the following non-default parameters: -c 3--smcogs--disable-embl. Annotated metagenomic markers were clustered using all vs. all diamond (https://github.com/bbuchfink/diamond) blastx. Blastx results were filtered using a python script requiring (a) E-value <1×10⁻⁵, (b) 90% coverage of length of coding sequence, and (c) >50% of coding sequences in a metagenomic marker present. Metagenomic markers were then grouped using markov clustering, resulting in a dereplicated library of 8,211 representative metagenomic markers identified in healthy human gut metagenomes.

### B. EMP Selection

A subset of metagenomic markers prevalent in healthy cohorts, referred to as essential microbial products (EMPs), were identified by clustering metagenomic markers presence/absence across 592 healthy patients from various geographic, genetic, and lifestyle backgrounds. Clusters with a mean prevalence >0.7 and z-score >10 within cohort were selected. To take sample imbalance into account, a proportion test was performed to assess stability across cohorts. This resulted in 1321 EMPs. For diagnostic analyses, metagenomic markers were subsetted from the full dataset, resulting in 1171 EMPs. Metagenomic markers were further subsetted, resulting in 590 EMPs.

### Metagenomic Data

Raw metagenomic reads from Ananthakrishnan et al., Cell Host Microbe, 21(5): 603-610.e3, 2017 were made available via ASPERAR transfer from the Broad Institute. There were 83 baseline (week 0, pretreatment) samples (43 Crohn's disease and 40 ulcerative colitis), 81 of which had baseline metagenomes available. The Ananthakrishnan et al. study presents the results of an analysis of disease activity and stool metagenomes in patients having Crohn's disease (CD) or ulcerative colitis (UC) who were treated with the anti-integrin therapy vedolizumab.

### C. Quantification of Metagenomic Markers Across Cohorts

To quantify metagenomic marker abundance across metagenomes, raw forward reads were mapped to the library of 8,211 metagenomic markers using diamond as described above. Normalized abundance of metagenomic markers was calculated such that abundance equals number of reads mapping divided by cumulative length of coding sequences in kilobase pairs divided by number of raw reads mapped.

### D. Data Preprocessing

EMP abundance data were transformed using rank-based inverse normal transformation (Blom), and centered and scaled.

### E. Modeling

Lasso models of the form treatment response at week 54˜gender+diagnosis+EMPs at baseline were fit using leave-one-out cross-validation with the glmnet package in R. Area under the receiver operating characteristic (ROC) curve was calculated using the pROC package in R, and the p-value of the ROC curve was computed using the Mann-Whitney method as previously described. The model achieved a sensitivity of 75% and a specificity of 75% at the point of maximum discrimination. These metrics were used for a post-hoc analysis of trial results from Sands et al., N Engl J Med, 381:1215-1226, 2019 to determine that 58% of patients selected using our model would have been responsive to treatment.

### F. Conclusions

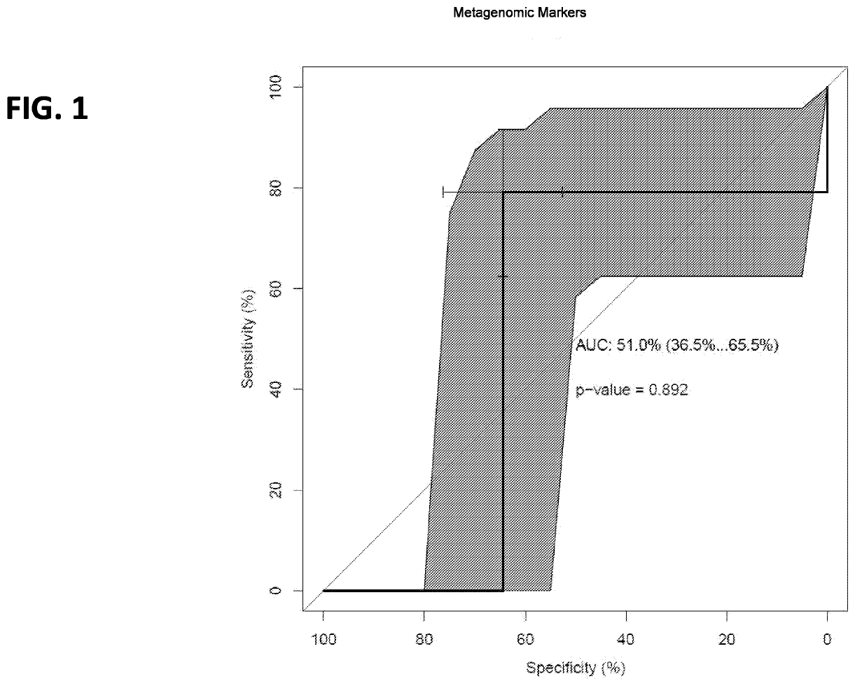

A set of 1171 metagenomic markers (SEQ ID NOs: 1-1171) were found to be significant predictors in a model discriminating between IBD (CD and UC) patients who were likely to be responders vs. non-responders to treatment with the anti-integrin therapy vedolizumab (FIG. 1).

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A method of identifying a subject having an inflammatory bowel disease (IBD) who is likely to respond to treatment with an anti-integrin therapy or other IBD therapy, the method comprising determining a level of one or more of SEQ ID NOs: 1-1171 in a sample from the subject, wherein a level of one or more of SEQ ID NOs: 1-1171 that is changed relative to a respective reference level for SEQ ID NOs: 1-1171 indicates that the subject is likely to respond to treatment with the anti-integrin or other IBD therapy.

2. A method for determining that a subject having an IBD is likely to respond to treatment with an anti-integrin therapy or other IBD therapy, comprising the steps of:
(a) measuring the nucleic acid level of one or more of SEQ ID NOs: 1-1171 in a sample collected from the subject using amplification and one or more pairs of primers specific for each of the one or more of SEQ ID NOs: 1-1171; and
(b) using the amplification results to determine whether the level of one or more of SEQ ID NOs: 1-1171 is changed relative to a respective reference level for SEQ ID NOs: 1-1171, thereby determining that the subject is likely to respond to treatment with an anti-integrin therapy or other IBD therapy.

3. The method of claim 1 or 2, wherein a level of one or more of SEQ ID NOs: 1-1171 is changed relative to the respective reference level for SEQ ID NO: 1-1171 in the sample from the subject and the method further comprises administering an anti-integrin or other IBD therapy to a subject.

4. The method of any one of claims 1-3, wherein the method comprises determining or measuring a level of at least two, at least three, at least four, at least five, at least six, at least seven, at least eight, at least nine, at least ten, at least fifteen, at least 20, at least 30, at least 40, at least 50, at least 60, at least 70, at least 80, at least 90, at least 100, at least 110, at least 120, at least 130, at least 140, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, or at least 1150 of SEQ ID NOs: 1-1171 in the sample from the subject.

5. The method of claim 4, wherein the method comprises determining or measuring a level of all 1171 of SEQ ID NOs: 1-1171 in the sample from the subject.

6. The method of any one of claims 1-5, wherein the anti-integrin therapy targets integrin 04B7.

7. The method of claim 6, wherein the anti-integrin therapy is vedolizumab.

8. The method of any one of claims 1-7, wherein the method further comprises treatment comprising fecal microbiota transplantation (FMT).

9. The method of any one of claims 1-8, wherein the reference level is a pre-assigned level.

10. The method of any one of claims 1-9, wherein the reference level is a level in a set of samples from a reference population.

11. The method of claim 10, wherein the reference population is a population of healthy subjects.

12. The method of any one of claims 1-11, wherein the level of each of SEQ ID NOs: 1-1171 that is determined or measured in the sample from the subject is a nucleic acid level.

13. The method of claim 12, wherein the nucleic acid level is a DNA level.

14. The method of any one of claims 1-13, wherein the change is a decrease relative to the reference level.

15. The method of claim 14, wherein the levels of at least 10%, at least 20%, at least 30%, at least 40%, at least 50%, at least 60%, at least 70%, at least 80%, at least 90%, or more than 90% of the sequences for which a level is determined or measured are decreased relative to a respective reference level for the sequence.

16. The method of any one of claims 1-13, wherein the change is an increase relative to the reference level.

17. The method of claim 16, wherein the levels of at least 10%, at least 20%, at least 30%, at least 40%, at least 50%, at least 60%, at least 70%, at least 80%, at least 90%, or more than 90% of the sequences for which a level is determined or measured are increased relative to a respective reference level for the sequence.

18. The method of any one of claims 1-17, wherein the sample comprises a sample of the microbiota of the subject.

19. The method of any one of claims 1-18, wherein the sample is a fecal sample.

20. A kit for identifying a subject having an inflammatory bowel disease (IBD) who is likely to respond to treatment with an anti-integrin or other IBD therapy, the kit comprising:
(a) polypeptides or polynucleotides capable of determining the level of one or more of SEQ ID NOs: 1-1171 in a sample from the subject; and
(b) instructions for use of the polypeptides or polynucleotides to determine the level of one or more of SEQ ID NOs: 1-1171 in a sample from the subject, wherein a level of one or more of SEQ ID NOs: 1-1171 that is changed relative to a respective reference level for SEQ ID NOs: 1-1171 indicates that the subject is likely to respond to treatment with the anti-integrin or other IBD therapy.

21. The kit of claim 20, wherein the kit comprises polypeptides or polynucleotides capable of determining the level of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or more than 99% of SEQ ID NOs: 1-1171 in a sample from the subject.

22. A method of identifying a subject having an inflammatory bowel disease (IBD) who is likely to respond to treatment with an anti-integrin or other IBD therapy, the method comprising determining a level of each of SEQ ID NOs: 1-1171 in a sample from the subject, wherein a level of at least 50% of SEQ ID NOs: 1-1171 that is changed relative to a respective reference level for SEQ ID NOs: 1-1171 indicates that the subject is likely to respond to treatment with an anti-integrin or other IBD therapy.

23. The method of claim 22, wherein a level of at least 50% of SEQ ID NOs: 1-1171 is changed relative to a respective reference level for SEQ ID NOs: 1-1171 in the sample from the subject and the method further comprises administering an anti-integrin or other IBD therapy to a subject.

24. The method of claim 22 or 23, wherein a level of at least 60%, 70%, 80%, 90%, or 95% of SEQ ID NOs: 1-1171 that is changed in a sample from the subject relative to a respective reference level for SEQ ID NOs: 1-1171 indicates that the subject is likely to respond to treatment with an anti-integrin or other IBD therapy.

25. The method of any one of claims 22-24, wherein the anti-integrin therapy targets integrin α₄β₇.

26. The method of claim 25, wherein the anti-integrin therapy is vedolizumab.

27. A method of treating a subject having an IBD using an anti-integrin therapy or other IBD therapy, wherein the subject was determined to be likely to respond to treatment with an anti-integrin therapy or other IBD therapy by the method of any one of claims 1-19 and 22-26.

28. A method of treating a subject having an IBD using an anti-integrin therapy or other IBD therapy, the method comprising the steps of:
(a) measuring the nucleic acid level of one or more of SEQ ID NOs: 1-1171 in a sample collected from the subject using amplification and one or more pairs of primers specific for each of the one or more of SEQ ID NOs: 1-1171;
(b) using the amplification results to determine whether the level of one or more of SEQ ID NOs: 1-1171 is changed relative to a respective reference level for SEQ ID NOs: 1-1171, thereby determining an increased likelihood of response to treatment with an anti-integrin therapy or other IBD therapy for the subject; and
(c) treating a subject who has been determined to have an increased likelihood of response to treatment with an anti-integrin therapy or other IBD therapy with the anti-integrin therapy or other IBD therapy.

29. The method of claim 28, wherein the nucleic acid level of the one or more of SEQ ID NOs: 1-1171 in the sample collected from the subject are measured using amplification and one or more pairs of primers specific for each of the one or more of SEQ ID NOs: 1-1171, and the amplification results are used to determine whether the level of one or more of SEQ ID NOs: 1-1171is changed relative to a respective reference level for SEQ ID NOs: 1-1171.

30. The method of claim 28 or 29, wherein the method comprises measuring the nucleic acid level of at least two, at least three, at least four, at least five, at least six, at least seven, at least eight, at least nine, at least ten, at least fifteen, at least 20, at least 30, at least 40, at least 50, at least 60, at least 70, at least 80, at least 90, at least 100, at least 110, at least 120, at least 130, at least 140, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, or at least 1150 of SEQ ID NOs: 1-1171 in the sample from the subject.

31. The method of claim 30, wherein the method comprises measuring the nucleic acid level of all 1171 of SEQ ID NOs: 1-1171 in the sample from the subject.

32. The method of any one of claims 28-31, wherein the reference level is a pre-assigned level.

33. The method of any one of claims 28-32, wherein the reference level is a level in a set of samples from a reference population.

34. The method of claim 33, wherein the reference population is a population of healthy subjects.

35. The method of any one of claims 28-34, wherein the nucleic acid level is a DNA level.

36. The method of any one of claims 28-35, wherein the change is a decrease relative to the reference level.

37. The method of claim 36, wherein the levels of at least 10%, at least 20%, at least 30%, at least 40%, at least 50%, at least 60%, at least 70%, at least 80%, at least 90%, or more than 90% of the sequences for which a level is measured are decreased relative to a respective reference level for the sequence.

38. The method of any one of claims 28-35, wherein the change is an increase relative to the reference level.

39. The method of claim 38, wherein the levels of at least 10%, at least 20%, at least 30%, at least 40%, at least 50%, at least 60%, at least 70%, at least 80%, at least 90%, or more than 90% of the sequences for which a level is measured are increased relative to a respective reference level for the sequence.

40. The method of any one of claims 28-39, wherein the sample comprises a sample of the microbiota of the subject.

41. The method of any one of claims 28-40, wherein the sample is a fecal sample.

42. A method of treating a subject having an IBD, the method comprising the steps of:
(a) measuring the nucleic acid level of each of SEQ ID NOs: 1-1171 in a sample collected from the subject, wherein a level of at least 50% of SEQ ID NOs: 1-1171 that is changed relative to a respective reference level for SEQ ID NOs: 1-1171 indicates that the subject is likely to respond to treatment with an anti-integrin therapy or other IBD therapy; and
(b) treating a subject who has been determined to be likely to respond to treatment with an anti-integrin therapy or other IBD therapy with the anti-integrin therapy or other IBD therapy.

43. The method of claim 42, wherein the nucleic acid levels of each of SEQ ID NOs: 1-1171 in the sample collected from the subject are measured using amplification and one or more pairs of primers specific for each of SEQ ID NOs: 1-1171, and the amplification results are used to determine whether the level of each of SEQ ID NOs: 1-1171 is changed relative to a respective reference level for SEQ ID NOs: 1-1171.

44. The method of any one of claims 26, 28, and 42, wherein the IBD therapy comprises an anti-integrin therapy.

45. The method of claim 44, wherein the anti-integrin therapy targets integrin α₄β₇.

46. The method of claim 44 or 45 wherein the anti-integrin therapy is vedolizumab.

47. The method of any one of claims 26, 28, and 42, wherein the IBD therapy comprises a biologic therapy, an anti-inflammatory agent, an antibiotic, or an immune system suppressor.

48. The method of claim 47, wherein the biologic therapy is vedolizumab (ENTYVIO®), infliximab (REMICADE®), adalimumab (HUMIRA®), golimumab (SIMPONI®), certolizumab (CIMZIA®), risankizumab (SKYRIZI®), or ustekinumab (STELARA®); the anti-inflammatory agent is a corticosteroid or an aminosalicylate; the antibiotic is ciprofloxacin (cipro) or metronidazole (Flagyl); or the immune system suppressor is azathioprine (AZASAN®, IMURAN®), mercaptopurine (PURINETHOL®, PURIXAN®), methotrexate (TREXALL®), tofacitinib (XELJANZ®), upadacitinib (RINVOQ®), or ozanimod (ZEPOSIA®).

49. The method of any one of claims 26, 28, 42, and 44 to 48, wherein the IBD therapy further comprises treatment by fecal microbiota transplant (FMT).
